(19) United States
(12) Patent Application Publication
(10) Pub. No.: US 2010/0024076 A1
(43) Pub. Date: Jan. 28, 2010

(54) ENHANCEMENT OF BIOMASS AND PRODUCTIVITY IN GRASSES

(75) Inventor: KELLY CRAVEN, Ardmore, OK (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: The Samuel Roberts Noble Foundation

(21) Appl. No.: 12/508,801

(22) Filed: Jul. 24, 2009

Related U.S. Application Data
(60) Provisional application No. 61/083,770, filed on Jul. 25, 2008.

(51) Int. Cl. A01H 5/00

(52) U.S. Cl. 800/301; 800/320; 800/302; 800/298

(57) ABSTRACT

The invention provides a synthetic combination of a Sebacina vermifera endophyte and a host grass plant, wherein the endophyte reproduces asexually and enhances the agronomic characteristics of the host plant. Methods for inoculating the host plant with the endophyte, for propagating the host-endophyte combination, and for detecting the presence of the endophyte and of its metabolites within a host plant are also described.

This application claims the priority of U.S. Provisional Application No. 61/083,770, filed on Jul. 25, 2008, the entire disclosure of which is incorporated herein by reference.

### BACKGROUND OF THE INVENTION

1. Field of the Invention

The invention relates to fungal endophytes (mycorrhizae) of host plants, such as the grass plants. In particular, the invention relates to Sebacina vermifera endophytes and to synthetic combinations of these endophytes with a host plant.

2. Description of the Related Art

Endophytes are fungal or bacterial organisms that live within plants. Fungal endophytes, such as mycorrhiza, survive within various host plant tissues, often colonizing the inter-cellular spaces of hosts leaves, stems, flowers or roots. These symbiotic endophyte-host relationships can provide fitness benefits to the host plant, such as enhancement of nutrition or chemical defense from potential herbivores. Root-colonizing mycorrhizae survive on photosynthetic carbohydrates from the plant, and in return, aid in the solublization and uptake of water and minerals to the host, which can lead to the promotion of seed germination and plant growth. Additionally, the association of a fungal endophyte with a host plant often provides protection from pathogens or tolerance to a variety of biotic and abiotic stresses, such as insect infestation, grazing, water or nutrient deficiency, heat stress, salt or aluminum toxicity, and freezing temperatures. Host growth and fitness promotion and protection are thought to be achieved through multiple beneficial properties of the endophyte-host association. For instance, the endophytic organisms may produce growth-regulating substances to induce biomass production and alkaloids or other metabolites that have anti-insect and anti-herbivore properties. Additionally, fungal endophytes may directly suppress or compete with disease causing microbes, protecting the plant from potential pathogens.

Endophytic relationships between non-pathogenic fungi and host grass plants are of agricultural interest. Switchgrass (Panicum virgatum) is a perennial warm season grass native to the North American Tallgrass Prairie and, along with other grasses, has been identified as a promising candidate for use as lignocellulosic biomass used for ethanol production. This is in large part due to the ability of this species to survive on marginal lands with limited agronomic inputs. Thus, there is a need to improve the production of biomass of grasses such as switchgrass as well as to promote host plant protection and tolerance from biotic and abiotic stresses.

### SUMMARY OF THE INVENTION

In one aspect, the invention provides a synthetic combination of a Sebacina vermifera endophyte and a host grass plant. In one embodiment, the endophyte is a S. vermifera strain selected from the group consisting of: MAFF305828, MAFF305830, MAFF305835, MAFF305837, MAFF305838 and MAFF305842. In another aspect the invention provides a S. vermifera endophyte in combination with a host grass plant such that the host grass plant displays increased biomass and/or vigor relative to a host grass plant of the same genotype that lacks the endophyte, when grown under the same conditions.

In certain embodiments, the host grass plant is artificially inoculated with the endophyte. In another embodiment, the endophyte protects the host grass plant from biotic stresses such as insect infestation, nematode infestation, and herbivore grazing, and/or abiotic stresses, such as water deficiency, nutrient deficiency, heat stress, salt toxicity, aluminum toxicity, heavy metal toxicity and freezing temperatures. The endophyte-host combination may be achieved by introduction of the endophyte to the host grass plant by a method selected from the group consisting of: inoculation, infection, grafting and combinations thereof. In certain embodiments, the host plant is a forage grass host plant. In another embodiment, the host plant is switchgrass (Panicum virgatum).

In a yet another aspect, the invention provides a seed comprising a S. vermifera endophyte. In one embodiment, the S. vermifera endophyte is provided into or onto the seed-coat of the seed. In still yet another aspect, the invention relates to a method for propagating a host grass plant-S. vermifera combination, comprising: a) obtaining a synthetic combination of a S. vermifera endophyte and a host grass plant and b) vegetatively reproducing the host grass plant tissue colonized by a S. vermifera endophyte.

In still yet another aspect, the invention provides a method for cultivating a host grass plant comprising: contacting the host grass plant with a S. vermifera endophyte, such that the endophyte colonizes the plant. In one embodiment, colonization of the host grass is achieved by introduction of the endophyte to the host grass plant by a method selected from the group consisting of: inoculation, infection, grafting, and combinations thereof. In another embodiment, the host grass plant has enhanced root growth, more tillers, enhanced total biomass, or enhanced seed yield relative to a host grass plant of the same genotype that lacks the endophyte, when grown under the same conditions. In yet other embodiments, the host grass plant displays tolerance to stress as relative to a host grass plant of the same genotype that lacks the endophyte, when grown under the same conditions. The stress may be selected from the group consisting of a biotic stress, a pest stress, an insect stress, an abiotic stress, and a water deficit stress. In one embodiment, the stress may be biotic stress caused by at least one organism selected from the group consisting of a mammalian or insect herbivore, or a microbial pathogen (nematode, fungus, bacteria, virus). In an additional embodiment, the stress is abiotic stress selected from the group consisting of: water deficiency, nutrient deficiency, heat stress, salt toxicity, aluminum toxicity, heavy metal toxicity, and freezing temperatures.

In still yet another aspect, the invention provides a method for cultivating a host grass plant comprising: contacting the host grass plant or a seed thereof with a filtrate of a cultured S. vermifera strain, wherein the plant has enhanced root growth, more tillers, enhanced total biomass, or enhanced seed yield or germination relative to a host grass plant of the same genotype that lacks the filtrate, when grown under the same conditions. In one embodiment, the host grass plant displays tolerance to stress relative to a host grass plant of the same genotype that lacks the endophyte, when grown under the same conditions, wherein the stress is selected from the group consisting of a biotic stress, a pest stress, an insect stress, an abiotic stress, and a water deficit stress.

In another aspect, the invention relates to a method for increasing the biomass of a plant comprising: contacting the host grass plant with a S. vermifera endophyte, such that the endophyte colonizes the plant, wherein the plant exhibits increased biomass relative to a host grass plant of the same genotype that lacks the endophyte, when grown under the same conditions.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein:

FIG. 1. S. vermifera associations with switchgrass genotype NF/GA-993 and barley roots in the first two weeks of inoculations as revealed by fluorescence microscopy. Abbreviations CON, SG and DAI denote control, switchgrass and days after inoculation, respectively.

FIG. 2. Florescent microscopy demonstrating the difference in growth patterns of Sebacina vermifera on switchgrass roots (A) and barley roots (B).

FIG. 3. Confocal images of associations between root of switchgrass genotype Alamo and S. vermifera (A-F) and an un-inoculated control (G) after two months of inoculation.

FIG. 4. Effect of S. vermifera strains on the plant height of switchgrass genotype Alamo at 45 DAI (days after inoculation). Error bars represent the standard error of mean.

FIG. 5. Photograph of the effect of S. vermifera strains on early growth of switchgrass seedling genotype Alamo at 45 DAI.

FIG. 6. Effect of filtrate of cultures of S. vermifera strains on seed germination of switchgrass genotypes Alamo and Kanlow.

FIG. 7. Photograph of the effect of S. vermifera strains on early growth of NF/GA-993 seedlings at two months after inoculation.

FIG. 8. Effect of S. vermifera inoculation on shoot and root length of switchgrass genotype NF/GA-993 at two months after inoculation. Bars with different letters are statistically different at 99% confident intervals.

FIG. 9. Effect of S. vermifera inoculation on shoot and root dry weight of switchgrass genotype NF/GA-993 at two months after inoculation. Bars with different letters are statistically different at 99% confident intervals.

### DETAILED DESCRIPTION OF THE INVENTION

The invention provides a synthetic combination of a Sebacina vermifera with a grass plant. It was found that this combination promotes plant growth, yielding at least a 12% increase in biomass, as demonstrated in the Examples below. In particular, this strong yield increase was observed in multiple genotypes of switchgrass (Panicum virgatum), which has been identified as a candidate for the production of bioethanol. As the need for biofuels increases, so does the need for more productive crops and methods to increase the yield of candidate grasses. The current invention provides such methods through the S. vermifera-grass plant combination.

In addition to the increased yields observed, the endophytic S. vermifera-grass plant combination of the invention demonstrates an increase in seed germination, plant fitness and stress tolerance. These benefits may also contribute to increased biomass production.

The basidiomycetous species S. vermifera is characterized primarily as an asexually reproducing mycorrhizae isolated from orchids, for instance Australian orchids (Warcup, 1988). Phylogenetic analysis has placed these endophytes within the order Sebacinales, based upon a maximum likelihood phylogenetic analysis of nuclear rDNA encoding the ribosomal large subunit (Deshmukh et al., 2006 and Weiss et al., 2004).

In particular embodiments, the S. vermifera endophyte may be of a strain selected from the group consisting of: MAFF305828, MAFF305830, MAFF305835, MAFF305837, MAFF305838 and MAFF305842. These strains are deposited with and maintained by the National Institute of Agrobiological Sciences Independent Administrative Institution (NIAS) in Tsukuba, Ibaraki, Japan. Additional S. vermifera strains available from the NIAS include MAFF305827, MAFF305829, MAFF305831, MAFF305832, MAFF305833, MAFF305834, MAFF305839, MAFF305840, and MAFF305841. S. vermifera strains have also been isolated from orchid and non-orchid host plants and are known to one of skill in the art (Warcup, 1988).

S. vermifera colonizes the roots of plants and forms hyphal networks on and inside the root cells and inter-cellular spaces, primarily proliferating in the dead cortical cells. These hyphae extend into the nearby rhizosphere and can form chlamydospores (survival structures) that may persist in soil upon decay of roots or root hairs. S. vermifera strains can therefore be isolated from soil or host plants which the fugal endophyte is colonizing using isolation methods known in the art.

In another aspect, the invention further provides a combination (also termed a “symbiotum”) of a host plant and a S. vermifera endophyte that allows for improved agronomic properties of host plants. The combination may be achieved by artificial inoculation, application, or other infection of a host plant, such as a grass plant, or host plant tissues with a S. vermifera strain of the present invention. Thus, a combination achieved by such an inoculation is termed a “synthetic” combination. The fungal endophyte may be present in intercellular spaces within plant tissue, such as the root. Its presence may also occur or may also be maintained within a grass plant or plant population by means of grafting or other inoculation methods.

These endophytes may also be introduced or maintained by such procedures, into various grasses, such as wheat (Triticum aestivum), durum wheat (Triticum turgidum ssp. durum), tall wheatgrass (Thinopyrum ponticum), western wheatgrass (Pascopyrum smithii), maize (Zea mays), rice (Oyrza sativa), sorghum (Sorghum bicolor), meadow fescue (Festuca pratensis), tall fescue (Festuca arundinacea), cereal rye (Secale cereale), Russian wild rye (Psathyrostachys juncea), oats (Avena sativa), bermudagrass (Cynodon dactylon), Kentucky bluegrass (Poa pratensis), big bluestem (Andropogon gerardii), little bluestem (Schizachyrium scoparium), blue grama (Bouteloua gracilis), black grama (Bouteloua eriopoda), side-oat grama (Bouteloua curtipendula), johnsongrass (Sorghum halepense), buffalograss (Buchloe dactyloides), and creeping bentgrass (Agrostis stolonifera). In one embodiment, the host plant is defined as a monocot. In an additional embodiment, the host plant is a forage grass host plant or a cereal. In a particular embodiment, the host plant is a grass host plant such as switchgrass (Panicum virgatum).

In certain embodiments, the agronomic qualities may be selected from the group consisting of: increased biomass, increased tillering, increased root mass, increased flowering, increased seed yield, and enhanced resistance to biotic and/or abiotic stresses, each of these qualities being rated in relation to plants of the same genotype grown under the same conditions, and differing only with respect to the presence or absence of a fungal endophyte. The stresses may include, for instance, drought (water deficit), cold, heat stress, nutrient deficiency, salt toxicity, aluminum toxicity, heavy metal toxicity, grazing by herbivores, insect infestation, nematode infection, and fungal infection, among others. In a particular embodiment, the enhanced resistance is provided by the endophyte and protects the host plant from subsequent infection by other fungal diseases, such as root rot, powdery mildew, Fusarium blight, Pythium blight, leaf spot, rust and snow mold, among others. This resistance may allow for improved biomass or seed yield relative, for instance, to grass plants not colonized by a S. vermifera endophyte. In another embodiment, the invention may be defined as a grass plant seed in combination with a S. vermifera strain or coated with a S. vermifera strain of the present invention.

The invention also relates to methods for protecting grass plants from biotic or abiotic stress, by means of introducing a S. vermifera strain of the present invention into a grass plant, and propagating the plant-endophyte combination by vegetative means. Vegetative propagation of the plant allows for propagation of the combination since fungal propagules (e.g. mycelia, conidia, and chlamydospores) are present in or on plant tissue or may infect the plant tissue.

The invention also provides methods for detecting the presence of a S. vermifera endophyte of the present invention within a host plant. This may be accomplished, for instance, by isolation of total DNA from tissues of a potential plant-endophyte combination, followed by PCR, or alternatively, Southern blotting, western blotting or other method known in the art, to detect the presence of specific nucleic or amino acid sequences associated with the presence of a S. vermifera strain of the present invention (Selosse et al., 2007). Alternatively, biochemical methods such as ELISA, HPLC, TLC, or fungal metabolite assays may be utilized to determine the presence of a S. vermifera strain of the present invention in a given sample of grass plant tissue. Additionally, methods for identification may include microscopic analysis, such as root staining, or culturing methods, such as grow out tests or other methods known in the art (Deshmukh et al., 2006). In particular embodiments, the roots of a potential grass plant-endophyte combination may be stained with fungal specific stains, such as WGA-Alexa 488, and microscopically assayed to determine fungal root associates, as described below.

### Definitions

Biomass: The total mass or weight, at a given time, of a plant or population plants, usually given as weight per unit area. The term may also refer to all the plants or species in the community (community biomass).

Culture filtrate: Broth or media obtained from cultures inoculated with a strain of fungi and allowed to grow. The media is typically filtered to remove any suspended cells leaving the nutrients, hormones or other chemicals.

Endophyte: An organism living within a plant cell. An endophyte may refer to a fungal organism that may confer an increase in yield, biomass, resistance or fitness in its host plant. Fungal endophytes may occupy the intra-cellular or extra-cellular spaces of plant tissue, including the leaves, stems, flowers or roots.

Genotype: The genetic constitution of a cell or organism.

Host plant: Any plant in which an endophytic fungi colonizes.

Increased yield: An increase in seed weight, seed size, seed number per plant, seed number per unit area (i.e. seeds, or weight of seeds, per acre), bushels per acre, tons per acre, kilo per hectare. Additionally, the term may refer to an increase in plant height, number of internodes, grain size, amount of tillers, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other yield traits that can effect yield include, efficiency of germination (including germination in stressed conditions), growth rate (including growth rate in stressed conditions), composition of seed (starch, oil, protein) and characteristics of seed fill.

Phenotype: The detectable characteristics of a cell or organism, which characteristics are the manifestation of gene expression.

Regeneration: The process of growing a plant from a plant cell (e.g., plant protoplast, callus or explant).

Synthetic combination: A combination (also termed a “symbiotum”) of a host plant and an endophyte. The combination may be achieved for example, by artificial inoculation, application, or other infection of a host plant, such as a grass plant, or host plant tissues with an endophyte.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Maintenance of Grass Plants and Sebacina vermifera Strains

Explants from nodal propagation of six different genotypes of switchgrass namely, Alamo, Kanlow, NF/GA-993, NFSG06 22(II) summer, NFSG06-224 summer and NFSG06-Source 80Ni summer were regularly maintained by splitting and transferring plantlets into MS shooting medium. Cultures of fifteen strains of S. vermifera were obtained from the National Institute of Agrobiological Sciences Independent Administrative Institution (NIAS) in Tsukuba, Ibaraki, Japan. S. vermifera cultures were maintained by sub culturing on malt extract agar medium on a monthly basis.

### Example 2

### Inoculation of Switchgrass and Barley with Sebacina vermifera

Three inculcation techniques were used to inoculate switchgrass and barley with six Sebacina vermifera strains. For the first method, show to be most effective, S. vermifera strains were cultivated in malt-yeast-peptone (MYP) medium (aqueous solution of 7 g/liter malt extracts, 1 g/liter peptone and 0.5 g/liter yeast extracts) as static culture for three weeks at room temperature. Mycelia were harvested mycelia using Mira cloth and rinsed three times with sterile distilled water, followed by grinding and the addition of equal amount of 0.05% Tween-20 (w/v). Roots of two days old barley seedlings and up to 10 days old switchgrass seedlings were dipped in mycelium suspension for three hours, and transplanted onto sterile turface and bentonite clay conditioner mixture (2:1). Transplants were maintained in a glasshouse. Roots of barley genotype Golden Promise and switchgrass genotypes Alamo and NF/GA-993 were inoculated with this method.

In an additional technique, rooted nodal explants of switchgrass were transplanted into soil amended with fungal mycelium. Alternatively, explant shoots were planted on medium colonized by the fungus.

A total of 350 Alamo seedlings were inoculated with six different strains of S. vermifera, namely; MAFF305828, MAFF305830, MAFF305835, MAFF305837, MAFF305838 and MAFF305842, and water control. Fifty seedlings were inoculated with each strain of S. vermifera and were transplanted.

A total of 6 g of switchgrass seeds of cultivar NF/GA-993 were scarified, surface sterilized and plated onto 3.5% water agar for germination to produce a minimum of 3500 seedlings for inoculation. A total of 288 seedlings were inoculated with each strain of S. vermifera and transplanted. An additional 288 seedlings were maintained as un-inoculated controls.

### Example 3

### Detection of Sebacina vermifera on Plant Roots

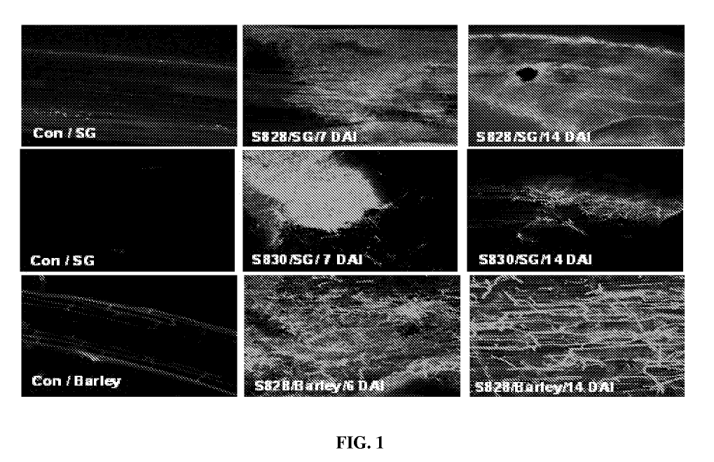

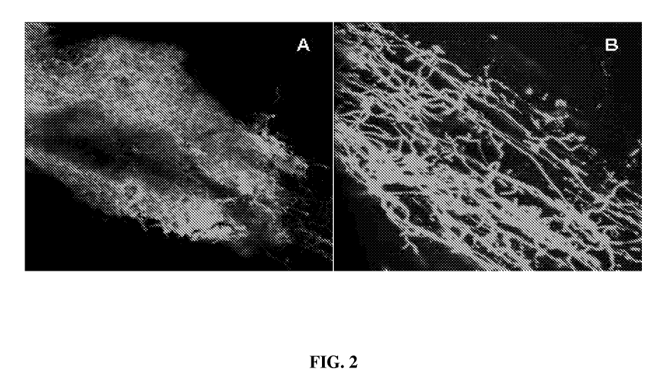

Roots of inoculated seedlings of barley and switchgrass were collected in weekly intervals and examined for fungal association using a propedium iodide and WGA-Alexa 488 mixture, and S. vermifera strains MAFF305828 and MAFF305830 were detected on the roots of barley genotype Golden Promise and switchgrass genotype NF/GA-993 up to four and seven weeks, respectively (FIG. 1). Microscopic observation showed different growth patterns of the fungus in switchgrass and barley roots (FIG. 2).

Staining of the root tissues with a mixture of propedium iodide and WGA-Alexa 488 (10 μg/ml of each) for 20 minutes followed by rinsing with PBS solution for 30 minutes produced reasonably good visualization of fungal-root associations under fluorescence microscope. Although propedium iodide was used here to stain the host tissues, it also stained fungal mycelium. Similarly, WGA-Alexa 488 which is expected to stain fungal tissue on the counter-stained host tissue, also stained host tissues to some levels. Therefore, other dyes, such as Congo red, may be used to counter-stain host tissue instead.

### Example 4

### Effects of Root Endophyte Sebacina vermifera on Switchgrass Growth

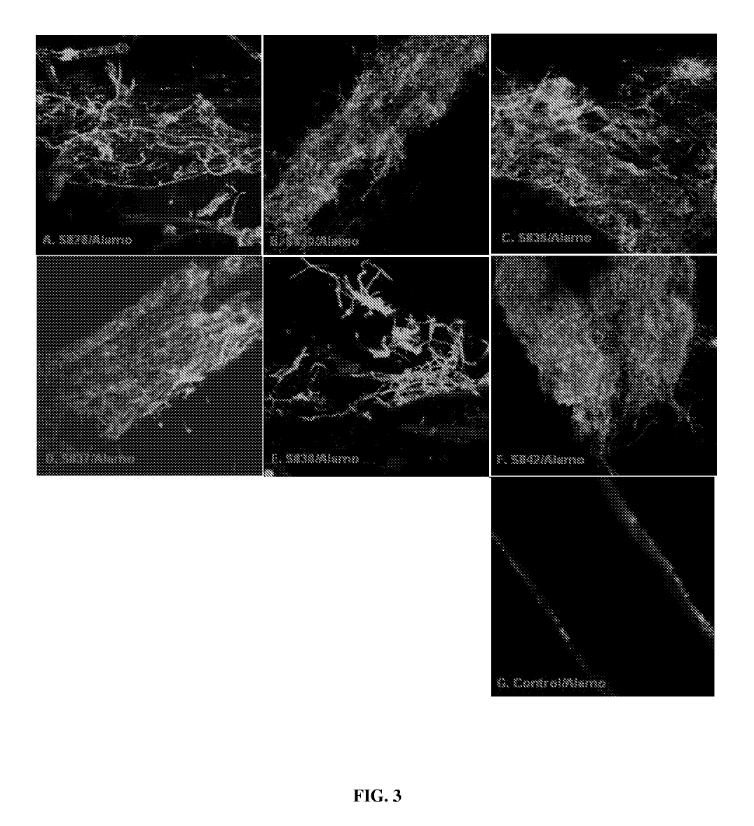

Alamo seedlings inoculated with different strains of S. vermifera were examined for the presence of S. vermifera on roots using confocal microscopy, and also monitored for effects on plant growth. The microscopic study at five week after inoculation (WAI) and at weekly intervals thereafter revealed that S. vermifera was consistently present on every inoculated plant root throughout the study period of 2 months (FIG. 3). Confocal microscopy with fluorescent dye WGA-Alexa 488 showed the presence of fungus exclusively on the inoculated roots, and its presence on other roots was rarely detected.

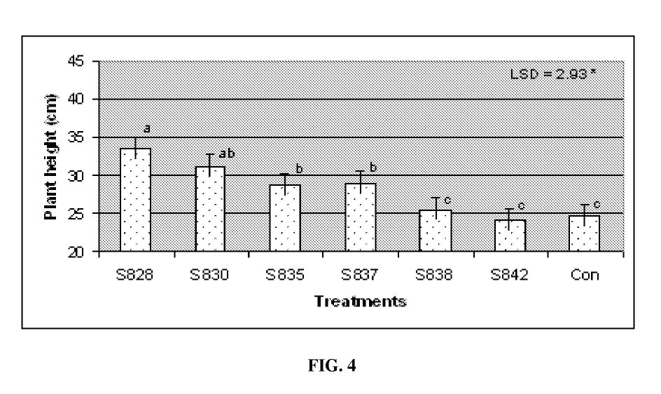

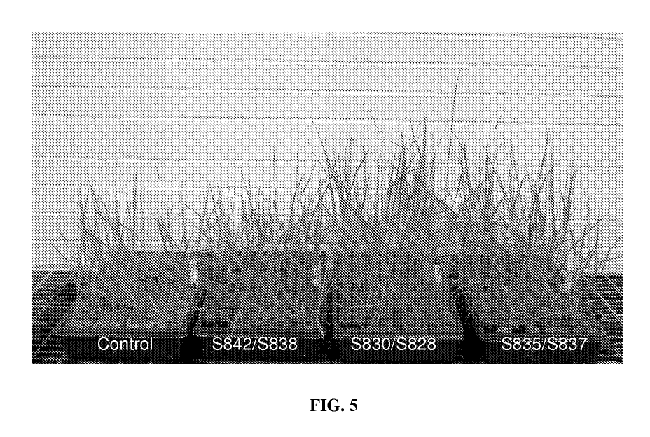

Phenotypic differences were obvious between plants inoculated with stains of S. vermifera and un-inoculated control. The plant mean height was computed based on at least 28 seedlings from each treatment at 45 days after transplanting and presented in FIG. 4. The phenotypic differences among the treatments is presented in FIG. 5. Two strains, MAFF305828 and MAFF305830 which had better colonization on inoculated switchgrass roots also had taller plants. The seedlings used in this experiment were only ten days old with a single root at the time of inoculation. The presence of fungus throughout the study period of two months and the noticeable impact on plant height suggests that inoculation of seedlings with developed root systems could facilitate a precise estimation of endophytic association on plant biomass production.

### Example 5

### Evaluation of Effects of Filtrate from Cultures of Sebacina vermifera Strains on Seed Germination

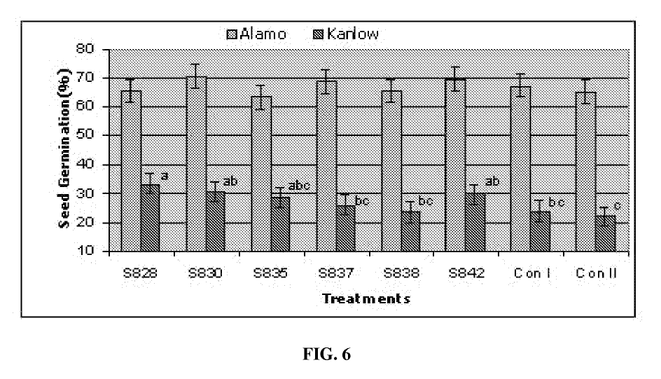

S. vermifera strains were cultivated in liquid malt-yeast-peptone (MYP) medium (aqueous solution of 7 g/liter malt extracts, 1 g/liter peptone and 0.5 g/liter yeast extracts) as static culture for three weeks at room temperature. Media was harvested and filtered through a 0.2 μm-pore-size filter to remove suspended cells. Switchgrass seeds of genotypes Kanlow and Alamo were plated on 2.5% water agar (WA) amended with filter sterilized MYP medium (100 seeds per plate). Seeds were incubated in the dark at 24° C. and germination was measured over several weeks. Microscopic detection revealed that some strains of S. vermifera enhance the germination by 12% over the control (FIG. 6).

### Example 6

### Evaluation of Effects of Sebacina vermifera Strains on Plant Growth and Biomass Production

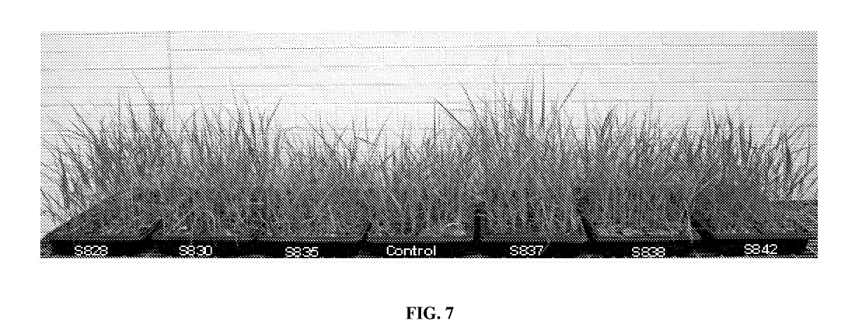

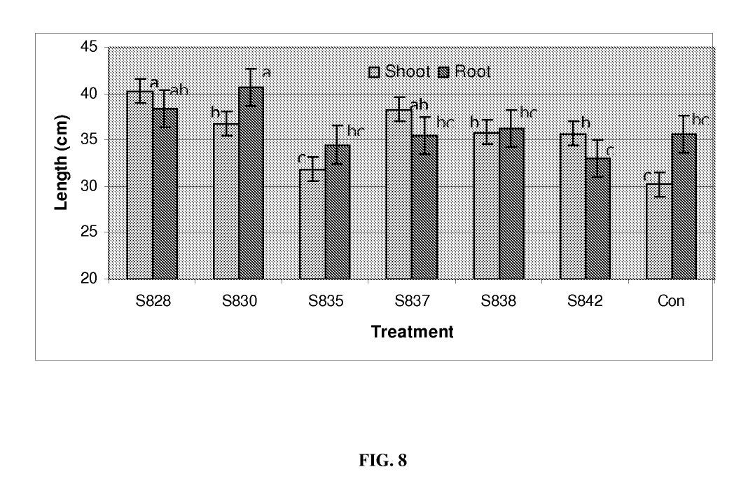

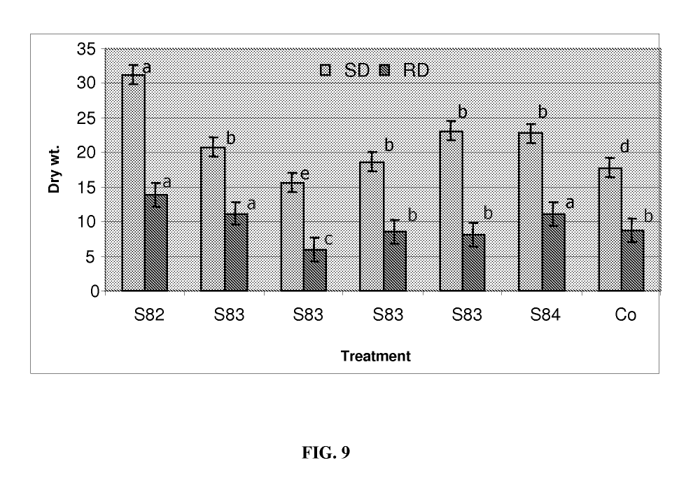

Switchgrass plants of genotype NF/GA-993 were inoculated with each of the six S. vermifera strains and a water control as described in Example 2. FIG. 7 depicts the effects of S. vermifera strains on early growth of NF/GA-993 seedlings at two months after inoculation. Plants were harvested two months after inoculation and shoot and root length and dry weight were determined. Results are shown in FIGS. 8 and 9.

In a separate experiment, 144 clonally propagated seedlings of switchgrass genotype VS-16 with well developed root systems were inoculated. Inoculated roots will be examined microscopically at fortnightly intervals and their impact on plant growth will also be assessed.

### Example 7

### Evaluation of the Frequency of Association of Sebacina vermifera Strains and Switchgrass

A total of 276 switchgrass seedlings of genotype NF/GA-993 were inoculated with six S. vermifera strains by the methods described in Example 2. Seedlings were microscopically examined for fungal association two months after inoculation. The results are shown in Table 1.

TABLE 1
Association Frequencies Between Switchgrass Seedlings of Genotype
NF/GA-993 and Sebacina vermifera Two Months After Inoculation

| | Number of Seedlings | | Percent Fungal |
|---|---|---|---|
| Strains | Examined | With Association | Association |
| MAFF305828 | 48 | 48 | 100 |
| MAFF305830 | 48 | 46 | 96 |
| MAFF305835 | 48 | 48 | 100 |
| MAFF305837 | 48 | 47 | 98 |
| MAFF305838 | 48 | 48 | 100 |
| MAFF305842 | 48 | 45 | 94 |
| Control | 48 | 0 | 0 |

### Example 8

### Evaluation of Effects of Sebacina vermifera Strains on Biotic and Abiotic Stress

Roots of switchgrass plants will be inoculated with each of the six S. vermifera strains. Plants will be exposed to multiple biotic and abiotic stresses, including, for instance drought (water deficit), cold, heat stress, nutrient deficiency, high salt levels, high levels of aluminum and heavy metals, grazing by herbivores, insect infestation, nematode infection, and fungal infection. Stress tolerance will be evaluated for each strain.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references listed below are incorporated herein by reference to the extent that they supplement, explain, provide a background for, or teach methodology, techniques, and/or compositions employed herein.
U.S. Pat. No. 6,111,170
International PCT Publication WO 99/29177
Baldi A, Jain A, Gupta N, Srivastava A K, Bisaria V S., 2008, Co-culture of arbuscular mycorrhiza-like fungi (Piriformospora indica and Sebacina vermifera) with plant cells of Linum album for enhanced production of podophyllotoxins: a first report. Biotechnol Lett. Epub ahead of print.
Barazani O, Benderoth M, Groten K, Kuhlemeier C, Baldwin I T, 2005. Piriformospora indica and Sebacina vermifera increase growth performance at the expense of herbivore resistance in Nicotiana attenuata. Oecologia 146(2):234-43.
Barazani O, von Dahl C C, Baldwin I T, 2007. Sebacina vermifera promotes the growth and fitness of Nicotiana attenuata by inhibiting ethylene signaling. Plant Physiol. 144(2):1223-32.
Bouton J H, Latch G C M, Hill N S, Hoveland C S, McCann M A, Watson R H, Parish J A, Hawkins L L, Thompson F N, 2002. Reinfection of tall fescue cultivars with non-ergot alkaloid-producing endophytes. Agronomy Journal 94: 567-574.
Deshmukh S, Hückelhoven R, Schäfer P, Imani J, Sharma M, 1 Weiss M, Waller F, Kogel K H, 2006. The root endophytic fungus Piriformospora indica requires host cell death for proliferation during mutualistic symbiosis with barley. Proc. Natl. Acad. Sci. 103: 18450-18457.
Hahn N, Varma A, Oelmüller R, Sherameti I, Members of Sebacinales Confer Resistance Against Heavy Metal Stress in Plants, 2007. In: Soil Biology, Volume 11, Advanced Techniques in Soil Microbiology, Varma, A, and Oelmüller, R., editors. 2007, 355-361.
Selosse M A, Setaro S, Glatard F, Richard F, Urcelay C, Weiss M., 2007. Sebacinales are common mycorrhizal associates of Ericaceae. New Phytol. 174(4):864-78.
Waller F, Mukherjee K, Deshmukh S D, Achatz B, Sharma M, Schäfer P, Kogel K H, 2008. Systemic and local modulation of plant responses by Piriformospora indica and related Sebacinales species. J Plant Physiol. 165(1):60-70.
Warcup J H, 1988. Mycorrhizal associations of isolates of Sebacina vermifera. New Phytologist, 110(2): 227-231.
Weiss M, Selosse M A, Rexer K H, Urban A, and Oberwinckler F, 2004. Sebacinales: a hitherto overlooked cosm of heterobasidiomycetes with a broad mycorrhizal potential. Mycol. Res. 108: 1003-1010.
Varma A, Verma S, Sudha, Sahay N, Bütehorn B, and Franken P, 1999. Piriformospora indica, a Cultivable Plant-Growth-Promoting Root Endophyte. Applied and Environmental Microbiology 65(6):2741-2744.
Verma S, Varma A, Rexer K, Hassel A, Kost G, Sarbhoy A, Bisen P, Butehorn B, and Franken P, 1998. Piriformospora indica, gen. et sp. nov., a New Root-Colonizing Fungus. Mycologia, 90(5):896-903.

What is claimed is:

1. A synthetic combination of a Sebacina vermifera endophyte and a host grass plant.

2. A Sebacina vermifera endophyte in combination with a host grass plant according to claim 1, wherein the host grass plant displays increased biomass and/or vigor relative to a host grass plant of the same genotype that lacks the endophyte, when grown under the same conditions.

3. The combination of claim 2, wherein the host grass plant is artificially inoculated with the endophyte.

4. The combination of claim 2, wherein the endophyte protects the host grass plant from biotic and/or abiotic stresses.

5. The combination of claim 4 wherein the abiotic stress is selected from the group consisting of: water deficiency, nutrient deficiency, heat stress, salt toxicity, aluminum toxicity, heavy metal toxicity and freezing temperatures.

6. The combination of claim 4 wherein the biotic stress is selected from the group consisting of: insect infestation, nematode infestation, and herbivore grazing.

7. The combination of claim 1, wherein the combination is achieved by introduction of the endophyte to the host grass by a method selected from the group consisting of: inoculation, infection, grafting and combinations thereof.

8. The combination of claim 1, wherein the host plant is a forage grass host plant.

9. The combination of claim 1, wherein the host plant is Panicum virgatum.

10. The combination of claim 1, wherein the endophyte is a Sebacina vermifera strain selected from the group consisting of: MAFF305828, MAFF305830, MAFF305835, MAFF305837, MAFF305838 and MAFF305842.

11. A seed comprising the endophyte of claim 1.

12. The seed of claim 11, wherein the endophyte is provided into or onto the seed-coat of the seed.

13. A method for propagating a host grass plant-Sebacina vermifera combination, comprising:
a) obtaining a synthetic combination of a Sebacina vermifera endophyte and a host grass plant of claim 1; and
b) vegetatively reproducing the host grass plant tissue colonized by a Sebacina vermifera endophyte.

14. A method for cultivating a host grass plant comprising: contacting the host grass plant with a Sebacina vermifera endophyte, such that the endophyte colonizes the plant.

15. The method of claim 14, wherein the host grass plant has enhanced root growth, more tillers, enhanced total biomass, or enhanced seed yield relative to a host grass plant of the same genotype that lacks the endophyte, when grown under the same conditions.

16. The method of claim 14, wherein the host grass plant displays tolerance to stress as relative to a host grass plant of the same genotype that lacks the endophyte, when grown under the same conditions.

17. The method of claim 16, wherein said stress is selected from the group consisting of a biotic stress, a pest stress, an insect stress, an abiotic stress, and a water deficit stress.

18. The method of claim 16, wherein the stress is biotic stress caused by at least one organism selected from the group consisting of a mammalian herbivore, a microbial pathogen and an insect.

19. The method of claim 16, wherein the stress is abiotic stress selected from the group consisting of: water deficiency, nutrient deficiency, heat stress, salt toxicity, aluminum toxicity, heavy metal toxicity, and freezing temperatures.

20. The method of claim 14, wherein colonization of the host grass is achieved by introduction of the endophyte to the host grass by a method selected from the group consisting of: inoculation, infection, grafting, and combinations thereof.

21. A method for cultivating a host grass plant comprising: contacting the host grass plant or a seed thereof with a filtrate of a cultured S. vermifera strain, wherein the plant has enhanced root growth, more tillers, enhanced total biomass, or enhanced seed yield or germination relative to a host grass plant of the same genotype that lacks the filtrate, when grown under the same conditions.

22. The method of claim 21, wherein the host grass plant displays tolerance to stress as relative to a host grass plant of the same genotype that lacks the endophyte, when grown under the same conditions, wherein the stress is selected from the group consisting of a biotic stress, a pest stress, an insect stress, an abiotic stress, and a water deficit stress.

23. A method for increasing the biomass of a plant comprising: contacting the host grass plant with a Sebacina vermifera endophyte, such that the endophyte colonizes the plant, wherein the plant exhibits increased biomass relative to a host grass plant of the same genotype that lacks the endophyte, when grown under the same conditions.
